(19) United States
(12) Patent Application Publication
(10) Pub. No.: US 2012/0157843 A1
(43) Pub. Date: Jun. 21, 2012

(54) METHOD AND SYSTEM TO SELECT SYSTEM SETTINGS AND PARAMETERS IN PERFORMING AN ULTRASOUND IMAGING PROCEDURE

(75) Inventors: Brent Jason Lavin, Wauwatosa, WI (US); Kimberly Canova, Wauwatosa, WI (US)

(73) Assignee: GENERAL ELECTRIC COMPANY, Schenectady, NY (US)

(21) Appl. No.: 12/970,400

(22) Filed: Dec. 16, 2010

(51) Int. Cl. A61B 8/14

(52) U.S. Cl. 600/443

(57) ABSTRACT

A selector system to select one of a series of transducer probes to perform an ultrasound imaging procedure is provided. The selector system includes a user interface, and a controller in communication to receive input and generate graphic illustrations at the user interface. The controller includes a processor to execute a series of programming instructions stored in a computer readable medium. The programming instructions instruct the processor to perform acts that include instructing the processor to request a series of input data at the user interface, receiving at the processor the series of input data from the interface, instructing the processor to automatically calculate a selection of one of the series of transducer probes in response to receiving the series of input data received at the user interface, and generating a graphic illustration of the selection of the one of the series of transducers to show at the user interface.

This invention generally relates to a method of and system for setting system parameters to perform an ultrasound imaging procedure, and method thereof.

### BACKGROUND

Known ultrasound imaging systems include an image processor used in conjunction with ultrasound beams emitted by a transducer probe to produce an ultrasound image on a display device. The display device presents the ultrasound image while the user interface permits an operator to control the functions, operations, image settings, adjustments to the ultrasound image, and the like.

Using current ultrasound systems on the market generally requires that the user have an extensive knowledge of ultrasound physics. The depth and accuracy of this knowledge can impact the quality and time for an ultrasound exam or procedure. It requires users to process an algorithm in their head based on the clinical scenario to know which transducer probe to select.

Users that may desire to use ultrasound imaging in emerging applications may not have in-depth technical knowledge of ultrasound physics, which may be a barrier to entry and lengthen the learning curve for using ultrasound.

U.S. Pat. No. 5,505,203 entitled “Method and System For Automatic Transducer Selection in Ultrasound Imaging System” describes a method and apparatus for activating one a plurality of already connected transducers accomplished by placing a proximity sensor at the selected transducer or holder thereof. However, this reference does not describe a guide for the users to automatically select which transducer best fits application in a given clinical scenario.

U.S. Pat. No. 5,615,678 entitled “Integral auto-selecting yoke/transducer connector for ultrasound transducer probe” describes an integral probe apparatus for ultrasound imaging which as a switch with one state when probe is seated in a yoke and a second state when probe is removed from the yoke. Again, this reference does not describe a guide for the user to automatically select a transducer that best fits application in a clinical scenario.

### BRIEF DESCRIPTION

The above-mentioned shortcomings, disadvantages and problems are addressed by the embodiments described herein in the following description of a method and system to calculate the appropriate transducer and system parameters and settings to use for a specific ultrasound imaging procedure. An advantage of the system provides that a pre-awareness is not needed as to which transducer and system parameters are most optimal to begin the ultrasound imaging procedure.

In one embodiment of the subject matter described herein, a selector system to select one of a plurality of types of transducer probes to perform an ultrasound imaging procedure is provided. The selector system comprises a user interface, and a controller in communication to receive input and generate graphic illustrations at the user interface. The controller includes a processor operable to execute a plurality of programming instructions stored in a computer readable medium. The programming instructions instruct the processor to perform acts that include instructing the processor to request a plurality of input data at the user interface, receiving at the processor the plurality of input data from the interface, instructing the processor to automatically calculate a selection of one of a plurality of transducer probes in response to receiving the plurality of input data received at the user interface, and generating a graphic illustration of the selection of the one of the plurality of transducers to show at the user interface.

In another embodiment of the subject matter described herein, a method is provided, comprising the acts of instructing a processor to request a plurality of input data at a user interface; receiving at the processor the plurality of input data from the interface; instructing the processor to automatically calculate a selection of one of a plurality of transducer probes in response to receiving the plurality of input data received at the user interface; and generating a graphic illustration of the selection of the one of the plurality of transducers to show at the user interface, the processor generating the graphic illustration of the selection in response to the input data received from the interface.

Another embodiment of a selector system in combination with an ultrasound imaging system having a plurality of transducer probes to acquire ultrasound image data of a patient is provided. The selector system comprises a user interface, and a controller in communication to receive input and generate graphic illustrations at the user interface. The controller includes a processor operable to execute a plurality of programming instructions stored in a computer readable medium. The programming instructions instruct the processor to perform acts including instructing the processor to request a plurality of input data at the user interface, receiving at the processor the plurality of input data from the interface, instructing the processor to automatically calculate a selection of one of a plurality of transducer probes in response to receiving the plurality of input data received at the user interface, generating a graphic illustration of the selection of the one of the plurality of transducers to show at the user interface, and instructing the ultrasound imaging system to activate in response to calculating the selection of one of the plurality of transducer probes to utilize in the medical procedure to perform.

Systems and methods of varying scope are described herein. In addition to the aspects and advantages described in this summary, further aspects and advantages will become apparent by reference to the drawings and with reference to the detailed description that follows.

### BRIEF DESCRIPTION OF THE DRAWINGS

FIG. 1 shows a schematic diagram of an embodiment of a selector system in combination with an ultrasound imaging system in accordance with the subject matter described herein.

FIG. 2 shows a schematic diagram of an embodiment of a method of operation of the selector system in conjunction with the ultrasound imaging system of FIG. 1.

FIG. 3 illustrates a schematic diagram an embodiment of a first graphic interface for illustration to a user interface of the selector system of FIG. 1.

FIG. 4 shows a schematic diagram of an embodiment of a second graphic interface for illustration to a user interface of the selector system of FIG. 1.

### DETAILED DESCRIPTION

In the following detailed description, reference is made to the accompanying drawings that form a part hereof, and in which is shown by way of illustration specific embodiments, which may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken in a limiting sense.

In this document, the terms “a” or “an” are used, to include one or more than one. In this document, the term “or” is used to refer to a nonexclusive or, unless otherwise indicated.

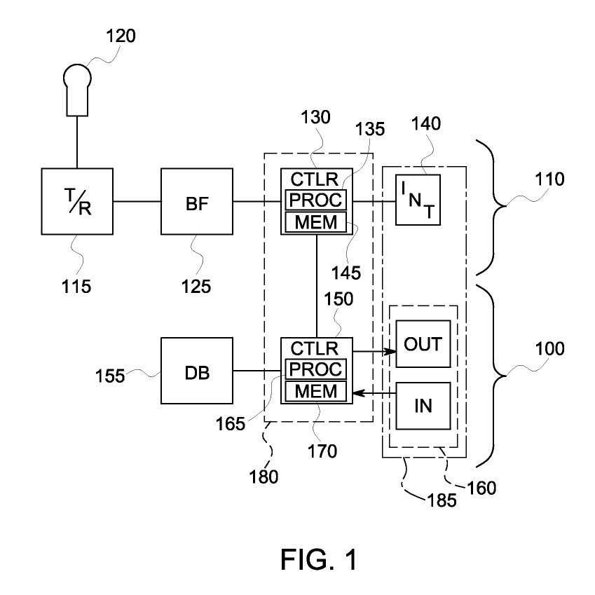

FIG. 1 illustrates an embodiment of a selector system 100 having a technical effect of providing a guide to a user in an automatic selection of a transducer and appropriate system parameters and settings of an ultrasound imaging system 110 to use for a specific ultrasound imaging procedure, in accordance to the subject matter described herein. A technical effect of the selector system 100 can be to enable the user to automatically select the transducer and system parameters and settings of the ultrasound imaging system 110 even though the user may have a low threshold level understanding or pre-awareness of ultrasound imaging or ultrasound physics.

The ultrasound imaging system 110 is generally operable to acquire real-time ultrasound imaging data of an imaged subject (not shown). Generally, the ultrasound imaging system 110 can include a transmitter/receiver 115 that drives an array of elements, for example, piezoelectric crystals, within a transducer, transducer probe or probe 120 to emit pulsed ultrasonic signals into a body or volume (not shown). A variety probes 120 and geometries transmitting the ultrasound signals from the probe may be used. Examples of probes 120 include a high-frequency linear transducer for superficial diagnostics or needle guidance, a low-frequency curved transducer for abdominal diagnostics, a phased array sector transducer for cardiac imaging, and a microconvex endocavitary probe for fetal diagnostics (female only.) The ultrasonic signals are back-scattered from structures in the body, for example, blood vessels or muscular tissue, to produce echoes that return to the elements of the probe 120 and received at the transmitter/receiver 115. The transmitter/receiver 115 communicates detection of the back-scattered ultrasound signals to the beamformer 125. The beamformer 125 generally performs beamforming including translating the echo data detected by the elements of the transducer 120 into ultrasound detection signal (e.g., RF). The beamformer 125 provides the ultrasound detection signal to a controller 130.

The controller 130 can include one or more multiple processors 135 operable to process and translate the ultrasound detection signal (e.g. RE signal or IQ data pairs) into a general real-time ultrasound image data for display on an interface or output device 140. One embodiment of the controller 130 may include a complex demodulator (not shown) that demodulates the ultrasound detection signal in an RF signal format to form IQ data pairs representative of the echo data. The RF or IQ signal data may then be provided directly to a computer-readable memory for storage (for example, temporary storage).

The controller 130 generally can include one or multiple processors 135 to receive the ultrasound detection signal from the beamformer 125. The one or multiple processors 135 in communication to execute computer-readable program instructions stored in a memory 145 of the controller 130 to perform translation of the ultrasound detection signal into an ultrasound image for display. The processor 135 can be adapted to perform one or more processing operations according to a plurality of selectable ultrasound modalities on the acquired ultrasound detection information. Acquired ultrasound detection information may be processed in real-time during a scanning session as the echo signals are received. Additionally or alternatively, the ultrasound detection information may be stored temporarily in the memory 145 during a scanning session and processed in less than real-time in a live or off-line operation. The acquired ultrasound detection data or information or signal not scheduled for display can immediately be stored in the memory 145. The memory 145 may comprise any known data storage medium, for example, a permanent storage medium, removable storage medium, and the like.

The controller 130 can be connected to the user interface 140 that may control some operations and be configured to receive inputs from an operator or user of the system 110. The interface 140 can include one or more monitors that present a graphic display of patient information, including diagnostic ultrasound images to the user for review, diagnosis and analysis. At least a portion of the interface 140 can include various types of input devices such as a mouse, keyboard, voice recognition, or a touch sensitive portion or touch sensitive screen to receive input from the user. The interface 140 may automatically display the generated ultrasound image data in various formats, for example, planes from two-dimensional (2D) and/or three-dimensional (3D) ultrasound data either in real-time or from stored 2D or 3D data-sets of ultrasound detection or image data in the memory 145. The processing of the ultrasound detection or image data can be based in part on user inputs, for example, user selections received at the user interface 140.

The system 100 can include a controller 150, a database 155, and an interface 160 in communication via a wireless or wired connection to the ultrasound imaging system 110. The controller 150 can include a processor 165 in communication to execute program instructions stored in a memory 170 in performing automatic selection or calculation of one of a plurality of transducer probes 120 and system parameters or settings prior to performing the ultrasound imaging procedure. The controller 150 can be connected in communication to store and recall selection of each of a series of transducer probes 120 and system parameters and settings stored for each of a plurality of input data, including (e.g., patient body type or mass representation, anatomical areas of interest for imaging, type of medical or imaging procedure, etc.) received at the user interface 160 in response to graphically illustrated prompts or requests for data to the user, as described in more detail below.

Although the system 100 is illustrated in FIG. 1 as independent of the ultrasound imaging system 110, the subject matter described herein should not be so limited. The system 100 or one or more portions thereof (including the controller 150, database 155 and interface 160) can be integrated with the ultrasound imaging system 110 or portions (including controller 130 and interface 140). For example, the controller 130 and controller 150 can be combined under a single controller 180. Likewise, the interface 140 can be combined with the interface 160 or portions thereof under a single interface 185.

Embodiments of the controllers 130, 150, 180 can be stand-alone computers (e.g., desktop or laptop, blackberry, etc.) or can include various arrangements or combinations of various types of processors (e.g., microprocessor, programmable logic controller, etc.) or combinations thereof in communication with various types of memory or computer readable mediums (e.g., memory stick, hard-drive, disk, CD, DVD, or other conventional storage medium or combination thereof). The interfaces 140, 160 and 185 can include input devices such as a keyboard, a touch-screen, a keypad, a joystick, dials, or other conventional input device or combination thereof operable to receive data from the user or clinician. The interfaces 140, 160 and 185 can also include output devices such as LCD or LED monitors, LEDs lights, touch-screens, alarm devices, etc. Examples of touch-screen technology can include but is not limited to touch sensitive elements such as capacitive sensors, membrane switches, and infrared detectors.

Having described a general construction of the embodiment of a system 100 in combination with the ultrasound system 110, the following is a general description of a method 200 of operation and technical effect of an embodiment of the selector system 100 in combination with the ultrasound 100 described above. Although the method 200 is described in accordance to the following acts, it should be understood that the sequence of the acts can vary. Also, it should be understood that the following description of acts is not limiting, and that one or more of the described acts may not be needed.

It should be understood that one or more of the following description of acts of the method 200 may be representative of modules of program instructions for execution by the processors 135, 165 of the controllers 130, 150 or 180.

In the following description of the method 200, the user is not required to know from previous experience or training which transducer probe 120 is the most optimal or best fit for the given medical or imaging procedure on the particular patient. Assume initially that the user has knowledge or is aware of general physical description (e.g., sex, body mass index (BMI) or body habitus or weight) of the patient and the medical or imaging procedure that the patient is to receive. However, the user does not have a threshold level of knowledge to select one of a plurality of types of transducer probes 120 to perform the ultrasound imaging procedure on the patient. Also assume that the database includes storage of a plurality of different types of transducer probes 120 and ultrasound imaging system parameters or settings that best fit application in performing one or more of a plurality of medical procedures for respective anatomical areas of interest and general parameters (e.g., gender, BMI or body habitus or weight) of the patient. Alternatively, the database can store algorithms that calculate a best fit of one of a plurality of different transducer probes 120 and system parameters and settings in response to received input of one of a plurality of types of medical or imaging procedures to be performed on one of a plurality of different parameters of the patient received at the user interface 160. The controller 150 can be connected in communication to recall selection of each of a series of transducer probes 120 and system parameters and settings stored in response to received user input or via application of the input data to the algorithms as described above, where the user input (e.g., patient body type or mass representation or body habitus, anatomical areas of interest for imaging, type of medical or imaging procedure, etc.) can be received at the user interface 160 in response to graphically illustrated prompts or requests for data to the user, as described in more detail below.

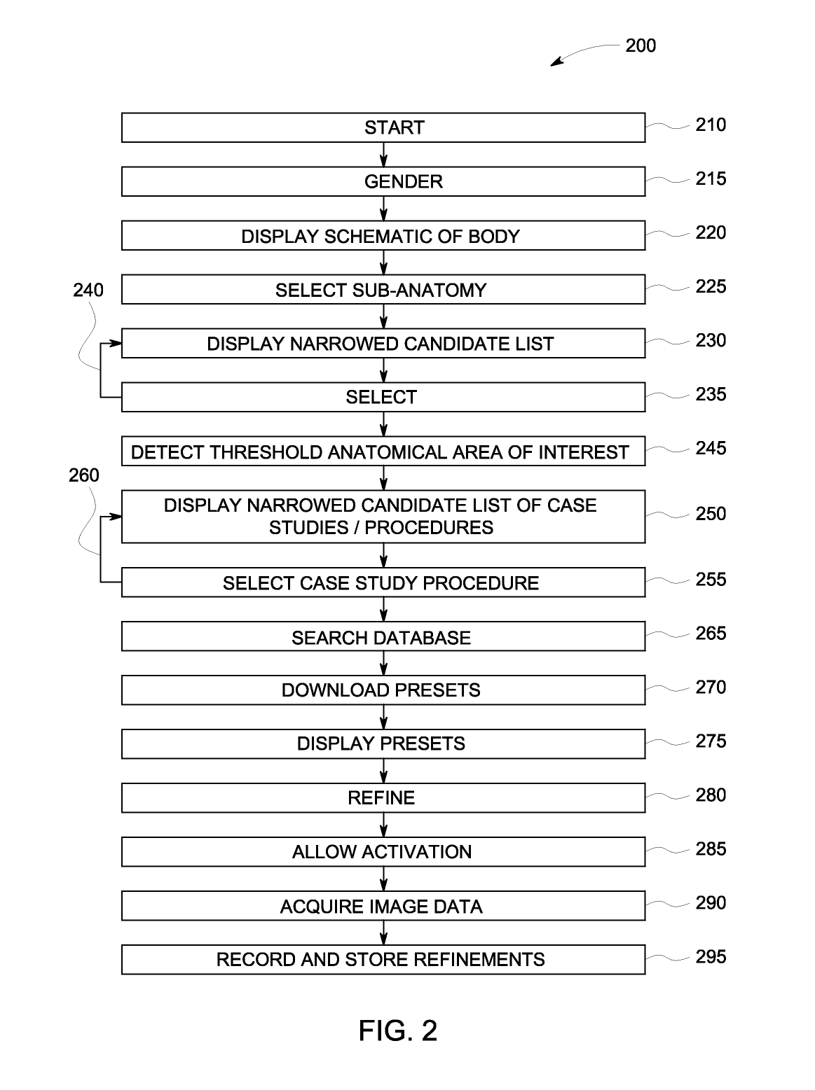

Referring now to FIG. 2, Act 210 includes a start of the method 200. Act 210 can include receiving an instruction from the user of a desire to initiate the process to automatically select one from a plurality of types of transducer probes 120 to employ in the requested medical or imaging procedure on the patient. Act 215 can include generating a graphic illustration and in response receiving user input data of a gender (e.g., male or female) of the patient. Act 220 can include generating a graphic illustration of human body for illustration to the user. Act 225 can include receiving a selection of anatomical area of interest (e.g., kidney, heart, upper extremity, vascular, gynecology, cardiac, etc.) of the patient correlated to a user input selection of a sub-anatomical area of interest of the patient. From this received user input, only anatomical areas of interest applicable to that gender are presented. Act 230 can include generating a graphic display or illustration of a narrowed selection of candidate list of anatomical areas of interest in response to the user selection in act 225. Act 235 can include receiving a selection from the narrowed candidate list of anatomical areas of interest from the user via the interface 160 or 185. Act 240 can include performing acts 230 and 235 for several repetitions in narrowing the user desired input of the anatomical area of interest to perform the medical or ultrasound imaging procedure on the patient.

Act 245 can include detecting a threshold level of detail of an anatomical area of interest so as to calculate or select one of the plurality of types of transducer probes to use.

Act 250 can include generating a graphic illustration or display of a narrowed candidate list of multiple case studies and medical procedures (i.e. shallow needle guidance, volume measurement, organ function, etc.) in response to the user input data of gender and anatomical area of interest received above. Act 255 can include receiving an input data of a selection from the graphic illustration of the narrowed candidate of multiple case studies and medical procedures. Act 260 can include repeating acts 250 and 255 to generate a graphic illustration and prompt the user to input selection of a desired narrowed level of selection of the case study or medical procedure to perform on the patient.

In acts 240 and 260 of the method 200 described above, graphic illustration of choices or candidate list of selections shown to the user can be based on previous inputs in acts 235 and 255, and can reduce a number of choices or list of multiple candidates selections and thereby reduce likelihood of confusion.

Act 265 can include instructing the controller 150, 180 or processor 165 therein to search the database 155 for the selection of one of the multiple types of transducers probes 120 and presets including system parameters and settings such as appropriate mode of ultrasound image acquisition (i.e. B-mode, Color Flow Mode, M-Mode, 3D scanning, real-time 3D imaging, volume scanning, 2D scanning with transducers having positioning sensors, scanning using 2D or matrix array transducers, and the like etc.) that best fits performing the case study or medical procedure on the patient, in comparison to other selections of types of transducer probes 120 and presets. For example, system parameters settings can include a depth of field of view in the ultrasound image, a gain of the ultrasound image, a frequency of the ultrasound waves emitted by the elements to obtain the ultrasound image, the focal position of the ultrasound waves emitted by the elements, and the imaging mode used to obtain the ultrasound image.

Act 270 includes downloading the transducer probe selection and presets from the database 155. Act 275 can include displaying the selection of the type of transducer probe 120 and presets to the user. Act 280 can include receiving input from the user of refinements to the selection of the transducer probe 120 or presets.

Act 285 can include instructing the controller 130, 180 to allow activation of the selected type of transducer probe and presets of the ultrasound imaging system 110 to perform the case study or medical procedure on the patient.

Act 290 can include acquiring the ultrasound image data of the patient for storage or display on the interface 140, 185. Act 295 can include receiving and recording refinements to the presets per instruction from the user for storage in the database 155 in response to the display of ultrasound image data on the interface 140, 185.

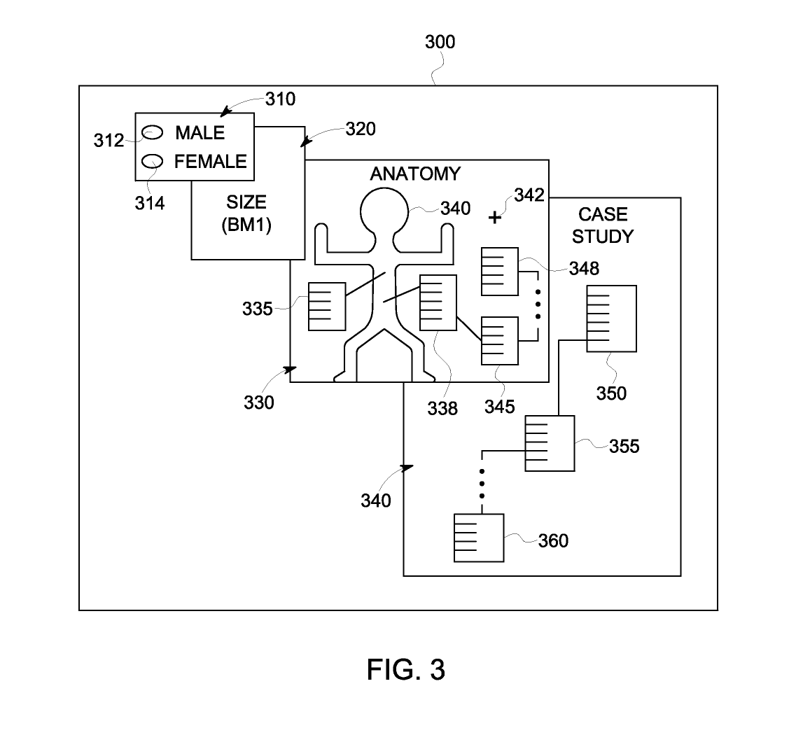

FIG. 3 illustrates an example screen shot or graphic illustration 300 in a of the user interface 160, 185 to the user in the method 200 of operating of the system 100 in combination with the ultrasound imaging system 110 as described above. The screenshot 300 according to one embodiment can provide touch screen functionality. Another embodiment of the screenshot 300 can be in a windows format operable to receive input from a user via a click of an input device such as a mouse device in manner known to those skilled in the art. The screenshot 300 may be presented on substantially all or a subpart of the viewable portion of the interface 160 or 185. While one particular layout of the screenshot 300 is shown in FIG. 3, other layouts, positions and orientations of the various components of the screenshot 300 are possible.

Referring to FIG. 3, a user selectable element 310 instructs the user to input a selection of the patient gender, as described in act 215 above. The illustrated user selectable element 310 includes a first user selectable element 312 correlated to a male and a second user selectable element 314 correlated to a female. Either a touch-screen activation or click of the input device (e.g., mouse device) on one of the user selectable elements 312 or 314 automatically generates communication of the input data of the patient gender to the selector system 100.

User selectable element 320 instructs the user to input a selection of a patient body mass index (BMI) or body habitus or weight. User selectable element 330 can instruct the user to input a selection of a patient anatomy and sub-anatomy of interest to perforin the case study or medical or imaging procedure. The user selectable element 320 can be operable to receive a numerical value of the BMI or body habitus or weight for communication to the selector system 100, or alternatively provide a plurality of candidates in a drop down menu format operable to be activated by a touch-screen functionality or via a click of an input device (e.g., mouse device) in a manner known in those skilled in the art.

An embodiment of the user selectable elements 330 can comprise a user selectable element of drop-down menus or lists 335, 338 of candidate selections or user selectable elements corresponding to an area of the graphically illustrated schematic of the body 340 that a cursor or other target 342 associated with the input device (e.g., mouse device) of the selector system 100. Each user selectable element of subcategories of narrowed drop down menus 345, 348 can be generated by the selector system 100 in response to the selection from the prior list of candidate selections 338 in the tree, such that selection from the user selectable elements 338 via the touch-screen functionality or input device click (e.g., mouse click) causes the system 100 to load and generate the subsequent user selectable elements 345, 348 of drop down menus or lists of candidate selections to choose from by the user. In a like manner to the embodiment of the user selectable elements 330, user selectable element 349 can include a graphic illustration of the user selectable elements 350, 355, and 360 of lists or menus of candidate case studies or medical or imaging procedures dependent in response to the selection of the user interface elements 310, 320 and 330. For example, the user selectable element 350 of the drop down list of candidate case studies correlated to a gynecology exam can be downloaded and generated for illustration on the screen shot 300 in response to selection of a female at the user element 310 having a selected habitus in element 320 and anatomy of interest in element 330, where such user element 350 of drop down list of the candidate case studies correlated to gynecology exam is not download or available for illustration at the screenshot 300 in response to receiving input data of the patient gender is male. Subsequent user selectable elements 350, 360 of menus or trees or lists of candidate selections can be downloaded and graphically illustrated to the user in the screenshot 300 for selection in response to the selection from the prior menu or list of candidate selections 350.

The screenshot 300 in FIG. 3 is described to include touch sensitive portions or user selectable elements correlated to the method 200 of operation of the system 100 in combination with the ultrasound imaging system 110. Each of the touch-sensitive portions can operate or act as a selection button or other interface capable of being touched by a user to provide user instructions of selections with respect to some aspect or feature or act of the method 200 of operation of the system 100 in conjunction with the ultrasound imaging system 110. Of course, the illustrated user selectable elements are shown merely as examples and should not be construed as global limitations on one or more embodiments described herein.

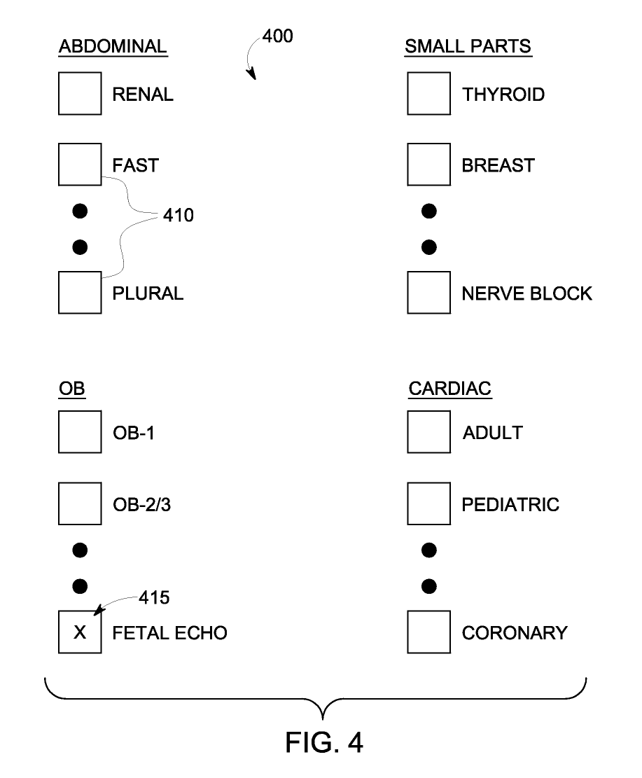

FIG. 4 illustrates an example embodiment of a utility interface display 400 operable to control the choices of anatomy or case studies shown/available to the user for selection via the one of the user interface elements 330 or 340 (See FIG. 3) at the interface 140 or 160. The example utility page 400 can include mouse activated, touch screen activated, or other input device actuated graphic interface elements 410 associated with different anatomy, case studies, or clinical applications of a particular probe 120. Each graphic interface element can be operable to receive instructions (see X and reference 415) from user in the selection or deselection of which anatomy or case studies or clinical procedures available for use with a particular probe 120. For example, the “X” can represent a selection of anatomy, case study, or clinical procedure for use with the particular probe 120. In another example, the “X” can represent a deselection of anatomy, case study, or clinical procedure not for use by the particular probe 120. With appropriate permissions to access the utility page 400, a technical effect of the utility page 400 can provide a tool for the user or an administrator to enable/disable certain clinical applications.

Although the above description of the system 100 and method 200 is described with reference to performance of an ultrasound imaging procedure on a subject, the systems 100 and method 200 may not limited to a healthcare application. The systems 100 and 200 can be employed in other applications of ultrasound imaging as well, such as security or industrial applications.

A technical effect of the system 100 and method 200 described above is to reduce the required user knowledge of ultrasound physics for selecting the appropriate transducer and system parameters to match the clinical scenario. Instead of requiring the user to know from their experience which specific transducer is going to meet their needs, this invention guides them through the process based on their input of the clinical situation. The technical effect of the above described system 100 and method 200 in automatic selection of one of the plurality of types of transducer probes and system parameters and settings can include no requirement of previous knowledge of ultrasound physics; reducing available choices for the user, avoiding confusion; reducing opportunity for error by standardizing procedures across the department; eliminating the concept of a preset to allow focus on the clinical procedure; optimizing the image parameters to show the product in the best possible circumstances; and reducing user dependence on product quality perception.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A selector system to select one of a plurality of types of transducer probes to perform an ultrasound imaging procedure, comprising:
a user interface; and
a controller in communication to receive input and generate graphic illustrations at the user interface, the controller including a processor operable to execute a plurality of programming instructions stored in a computer readable medium, the programming instructions to instruct the processor to perform acts including:
instructing the processor to request a plurality of input data at the user interface,
receiving at the processor the plurality of input data from the interface,
instructing the processor to automatically calculate a selection of one of the plurality of transducer probes in response to receiving the plurality of input data received at the user interface, and
generating a graphic illustration of the selection of the one of the plurality of transducers to show at the user interface.

2. The system of claim 1, further comprising an act of automatically instructing the processor to switch an ultrasound imaging system from an inactive state to an active state so as to perform ultrasound imaging acquisition.

3. The system of claim 1, wherein the act of instructing the processor to request the plurality of input data includes generating a first user selectable element to receive an input of a patient gender

4. The system of claim 3, wherein the act of instructing the processor to request the plurality of input data includes generating a second user selectable element to receive an input of a body habitus.

5. The system of claim 4, wherein the act of instructing the processor to request the plurality of input data includes generating a third user selectable element to receive an input of a patient anatomical area of interest, the third user selectable element including a first list of a plurality of candidate patient anatomical areas of interest for selection generated in response to the input of the patient gender.

6. The system of claim 5, wherein the act of instructing the processor to request the plurality of input data further includes generating a second list of a second plurality of candidate patient anatomical areas of interest for selection generated as a sub-category of the first list of the plurality of patient anatomical areas of interest, the second list automatically generated in response to receiving a selection of one of the plurality of anatomical areas of interests in the first list of the plurality of patient anatomical areas of interest.

7. The system of claim 5, wherein the act of instructing the processor to request the plurality of input data further includes generating a fourth user selectable element to receive an input of a medical procedure to perform, the fourth user selectable element including a first list of a plurality of medical procedures to perform, the first list of the plurality of medical procedures generated in response to receiving the input of the patient gender, the input of the patient body habitus, and the input of the patient anatomical area of interest.

8. The system of claim 6, wherein the act of instructing the processor to automatically calculate a selection of one of a plurality of transducer probes is dependent on the input of the patient gender, the input of the patient body habitus, the input of the patient anatomical area of interest, and the input of the medical procedure to perform.

9. The system of claim 8, wherein the programming instructions further include the act of the processor instructing an ultrasound imaging system to move from an inactive state to an active state in response to calculating the selection of one of the plurality of transducer probes to utilize in the medical procedure to perform.

10. A method comprising the acts of:
instructing a processor to request a plurality of input data at a user interface;
receiving at the processor the plurality of input data from the interface;
instructing the processor to automatically calculate a selection of one of a plurality of transducer probes in response to receiving the plurality of input data received at the user interface; and
generating a graphic illustration of the selection of the one of the plurality of transducer probes to show at the user interface, the processor generating the graphic illustration of the selection in response to the input data received from the interface.

11. The method of claim 10, further comprising an act of automatically instructing the processor to switch an ultrasound imaging system from an inactive state to an active state so as to perform ultrasound imaging acquisition.

12. The method of claim 10, wherein the act of instructing the processor to request the plurality of input data includes generating a first user selectable element to receive an input of a patient gender

13. The method of claim 12, wherein the act of instructing the processor to request the plurality of input data includes generating a second user selectable element to receive an input of a body habitus.

14. The method of claim 13, wherein the act of instructing the processor to request the plurality of input data includes generating a third user selectable element to receive an input of a patient anatomical area of interest, the third user selectable element including a first list of a plurality of candidate patient anatomical areas of interest for selection generated in response to the input of the patient gender.

15. The method of claim 14, wherein the act of instructing the processor to request the plurality of input data further includes generating a second list of a second plurality of candidate patient anatomical areas of interest for selection generated as a sub-category of the first list of the plurality of patient anatomical areas of interest, the second list automatically generated in response to receiving a selection of one of the plurality of anatomical areas of interests in the first list of the plurality of patient anatomical areas of interest.

16. The method of claim 14, wherein the act of instructing the processor to request the plurality of input data further includes generating a fourth user selectable element to receive an input of a medical procedure to perform, the fourth user selectable element including a first list of a plurality of medical procedures to perform, the first list of the plurality of medical procedures generated in response to receiving the input of the patient gender, the input of the patient body habitus, and the input of the patient anatomical area of interest.

17. The method of claim 16, wherein the act of instructing the processor to automatically calculate a selection of one of a plurality of transducer probes is dependent on the input of the patient gender, the input of the patient body habitus, the input of the patient anatomical area of interest, and the input of the medical procedure to perform.

18. The method of claim 17, wherein the programming instructions further includes the act of the processor instructing an ultrasound imaging system to transition from an inactive state to an active state in response to calculating the selection of one of the plurality of transducer probes to utilize in the medical procedure to perform.

19. A selector system in combination with an ultrasound imaging system having a plurality of transducer probes to acquire ultrasound image data of a patient, the selector system comprising:
a user interface; and
a controller in communication to receive input and generate graphic illustrations at the user interface, the controller including a processor operable to execute a plurality of programming instructions stored in a computer readable medium, the programming instructions to instruct the processor to perform acts including:
instructing the processor to request a plurality of input data at the user interface,
receiving at the processor the plurality of input data from the interface,
instructing the processor to automatically calculate a selection of one of the plurality of transducer probes in response to receiving the plurality of input data received at the user interface,
generating a graphic illustration of the selection of the one of the plurality of transducer probes to show at the user interface, and
instructing the ultrasound imaging system to activate in response to calculating the selection of the one of the plurality of transducer probes.

20. The selector system, further comprising a user interface element comprising multiple actuatable graphic interface elements operable to receive a user instruction to select or deselect from a predefined anatomy, a case study, and a clinical application so as to enable or disable respectively with use by each of the plurality of transducer probes.
